(19) United States
(12) Patent Application Publication
(10) Pub. No.: US 2005/0119198 A1
(43) Pub. Date: Jun. 2, 2005

(54) Novel target to inhibit angiogenesis

(76) Inventors: Peter Carmeliet, Blanden (BE); Lieve Moons, Herselt (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/502,244

(22) PCT Filed: Feb. 7, 2003
(86) PCT No.: PCT/EP03/01229

(30) Foreign Application Priority Data
Feb. 8, 2002 (EP) 02075544.3
Jul. 9, 2002 (EP) 02077742.1
Jan. 24, 2003 (EP) 03100148.0

(51) Int. Cl. A61K 31/70; A01N 43/04

(52) U.S. Cl. 514/43

(57) ABSTRACT

The invention relates to the field of angiogenesis. In particular the invention relates to the use of molecules binding to prominin-1 that can be used for the manufacture of a medicament to prevent pathological angiogenesis.

### FIELD OF THE INVENTION

The invention relates to the field of angiogenesis. In particular the invention relates to the use of molecules binding to prominin-1 that can be used for the manufacture of a medicament to prevent pathological angiogenesis.

### BACKGROUND OF THE INVENTION

Prominin-1 (PROM-1), also called AC133 or recently designed CD133 (National Center for Biotechnology, 2000), is a rather novel human hematopoietic stem cell antigen ¹ of unknown physiological or pathological function. Prominin-1-antigen was first detected on CD34^{bright} hematopoietic stem cells ² and has since been widely used to facilitate the analysis and isolation of hematopoietic and primitive cells ³⁻⁵. Only few prominin-1⁺ cells do not coexpress CD34: these cells are very small and define a population of unknown delineation ⁶. In acute myeloid leukemias, PROM-1 expression is often but not always associated with CD34 expression ^{7,8}. Prominin-1 is also found on acute lymphoid leukemia blasts and on a subset of CD34⁺ B-cell precursors ⁹. Flow cytometry analyses of a wide panel of human cell lines showed that only retinoblastoma and teratocarcinoma cell lines express prominin-1¹⁰. More recently, it was shown that endothelial progenitor cells co-express PROM-1 antigen and the endothelial cell-specific receptor kinase-inert domain-containing acceptor (KDR) in subpopulations of CD34⁺ cells derived from fetal liver, bone marrow, cord blood and peripheral blood ^{11,12}. Recently, human central nervous system stem cells were also reported to express prominin-1-antigen ¹³. A characteristic feature of this protein is its rapid down-regulation during cell differentiation ^{12,14}, which makes it a unique cell surface marker for the identification and isolation of stem cells and progenitor cells. Human PROM-1 antigen is a glycoprotein of 120 KD and contains an extracellular N-terminus, two extracellular loops, five transmembrane domains, two small cysteine-rich cytoplasmic loops and a cytoplasmic C terminus ¹. Recently a novel isoform of human PROM-1 with a 27 basepair deletion has been described ¹⁵. A structural and sequence-related protein, was identified as the mouse orthologue of human PROM-1 ¹⁴. The 5-transmembrane structure appears phylogenetically conserved from mammals to zebrafish and in fruit flies and nematodes ^{16,17}, Murine prominin-1, which has a 65% amino acid homology with human PROM-1 also exists in two isoforms. The short human and murine prominin isoform both encode proteins that lack a 9-amino acid segment at the same location in the N-terminal extracellular region just proximal to the first transmembrane domain ^{15,18,19}. Although human PROM-1 has been used as a cell surface marker to identify and isolate certain stem cell and progenitor cell populations, the molecular mechanism of how this protein functions remain unclear. The possible role of PROM-1 in hematopoiesis and vasculogenesis in the developing embryo and, after birth, in angiogenesis, postnatal vasculogenesis and hematopoietic stem cell trafficking, remains largely unknown. To study in detail the in vivo role of PROM-1 in the present invention PROM-1 deficient mice were generated. It was surprisingly found that PROM-1 has a key role in pathological angiogenesis and that inhibitors of PROM-1 can be used in therapeutic strategies to inhibit blood vessel formation in various pathological disorders.

### AIMS AND DETAILED DESCRIPTION OF THE INVENTION

The “hemangioblast” is a putative progenitor cell that has the potential to form either endothelial or hematopoietic cells. It exists during embryogenesis in the blood island region of the yolk sac ²⁰, which is therefore the earliest site of hematopoiesis and vasculogenesis. Until recently, vasculogenesis has been thought to be restricted to the yolk sac and the early embryogenesis. However, novel observations have revealed in adulthood a situation consistent with vasculogenesis: endothelial cells derived from angioblasts or “hemangioblasts” previously isolated from peripheral blood or bone marrow are incorporated into sites of neovascularization in physiological and pathological conditions ²¹⁻²⁵. In addition, the number of these endothelial cell progenitors increases in the peripheral blood during tissue ischemia or following the administration of VEGF or GM-CSF, a cytokine known to induce mobilization of hematopoietic stem cells from the bone marrow into the peripheral blood ^{24,25}. Recent studies in humans, dogs, rats, rabbits and mice have indeed indicated the presence of endothelial precursor cells (EPCs) in bone marrow and peripheral blood during adult life which can be mobilized and incorporated into newly formed vessels or are involved in endothelialization of implants ^{23,26-32}. Interestingly, in all these experiments, endothelial cell progenitors are isolated together with other hematopoietic stem cells by using antibodies directed against hematopoietic stem cell antigens. PROM-1 is expressed on lineage non-committed stem and progenitor cells but not on mature peripheral blood cells and umbilical vein derived endothelial cells ². CD34⁺ cells co-expressing VEGFR-2 and PROM-1, have been isolated from peripheral blood, cord blood, fetal liver and bone marrow. When grown in the presence of VEGF and FGF-2 or the cytokine stem cell growth factor (SCGF), these cells give rise to endothelial cells, thus suggesting that this subset of CD34⁺, VEGFR-2⁺ & CD133⁺ cells may play a role in neovasculogenesis 3,5,11. The present invention uses a transgenic mouse deficient in PROM-1 to study the involvement of PROM-1 in several pathological models of angiogenesis. For the sake of clarity the nucleotide sequence of human prominin-1 is designated here as SEQ ID NO: 1 and the amino acid sequence of human prominin-1 is designated as SEQ ID NO: 2. The present invention shows that inhibitors of prominin-1 can be used in therapeutic applications for the prevention of pathological angiogenesis.

Thus the invention provides in one embodiment the use of a molecule which comprises a region specifically binding to prominin-1 (SEQ ID NO: 2) or nucleic acids encoding prominin-1 (SEQ ID NO: 1), for the manufacture of a medicament to treat pathological angiogenesis.

According to the invention molecules that comprise a region specifically binding to prominin-1 or nucleic acids encoding prominin-1 which can be used for the manufacture of a medicament to treat pathological angiogenesis can be chosen from the list comprising an antibody or any fragment thereof binding to prominin-1, a (synthetic) peptide, a protein, a small molecule specifically binding to prominin-1 or nucleic acids encoding prominin-1 or a regulatory region (e.g. a promoter region) of prominin-1, RNA aptamers against prominin-1, a ribozyme against nucleic acids encoding prominin-1, anti-sense nucleic acids hybridising with nucleic acids encoding prominin-1 and small interference RNA's (siRNA) against prominin-1.

The wording ‘a molecule which comprises a region specifically binding to prominin-1 or nucleic acids encoding prominin-1’ relates (1) on the one hand to molecules binding to nucleic acids encoding prominin-1 or to regulatory genetic regions of prominin-1, said molecules inhibit the gene expression of prominin-1 (thus the inhibition of prominin-1 transcription and/or translation of a gene transcript (mRNA) of prominin-1 and (2) on the other hand to molecules that inhibit the activity of the prominin-1 protein. The inhibition of gene expression can be measured conveniently by methods known in the art such as for example RT-PCR analysis of the prominin-1 transcript or for example western blot analysis of the prominin-1 protein, said inhibition is preferably at least 20%, 30%, 40%, 50%, 60%, 70%, 80%, 90% or even higher. Measurement of molecules that bind to the prominin-1 protein and inhibit the activity of prominin-1 can for example be carried out by various methods for determining pathological angiogenesis as described in the examples of the present invention. Said inhibition of prominin-1 activity is preferably at least 20%, 30%, 40%, 50%, 60%, 70%, 80%, 90% or even higher. Thus in another embodiment the invention provides the use of a molecule that inhibits the expression and/or activity of prominin-1 for the manufacture of a medicament for treatment of pathological angiogenesis. In the latter embodiment activity relates to the gene product (the protein) and expression relates to the gene: mRNA formation and/or translation of the mRNA of prominin-1.

The term ‘antibody’ or ‘antibodies’ relates to an antibody characterized as being specifically directed against prominin-1 or any functional derivative thereof, with said antibodies being preferably monoclonal antibodies; or an antigen-binding fragment thereof, of the F(ab′)₂, F(ab) or single chain Fv type, or any type of recombinant antibody derived thereof. These antibodies of the invention, including specific polyclonal antisera prepared against prominin-1 or any functional derivative thereof, have no cross-reactivity to others proteins. The monoclonal antibodies of the invention can for instance be produced by any hybridoma liable to be formed according to classical methods from splenic cells of an animal, particularly of a mouse or rat immunized against prominin-1 or any functional derivative thereof, and of cells of a myeloma cell line, and to be selected by the ability of the hybridoma to produce the monoclonal antibodies recognizing prominin-1 or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′₂ and ssFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type.

In a specific embodiment the antibodies against prominin-1 can be derived from animals of the camelid family. In said family immunoglobulins devoid of light polypeptide chains are found. Heavy chain variable domain sequences derived from camelids are designated as VHH's. “Camelids” comprise old world camelids (Camelus bactrianus and Camelus dromaderius) and new world camelids (for example Lama paccos, Lama glama and Lama vicugna). EP0656946 describes the isolation and uses of camelid immunoglobulins and is incorporated herein by reference.

Small molecules, e.g. small organic molecules, and other drug candidates can be obtained, for example, from combinatorial and natural product libraries.

Also within the scope of the invention are oligoribonucleotide sequences, that include anti-sense RNA and DNA molecules and ribozymes that function to inhibit the translation of prominin-1 mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of the prominin-1 nucleotide sequence, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of prominin-1 RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize anti-sense RNA constitutively or inducibly, depending on the promoter used, can be introduced stably into cell lines.

In a particular embodiment short interference RNA molecules (siRNA) can be used for the manufacture of a medicament for treatment of pathological angiogenesis. Said interference RNA molecules can be generated based on the genetic sequence of prominin-1 (SEQ ID NO: 1). RNA interference (RNAi) is based on the degradation of particular target sequences by the design of short interference RNA oligo's (siRNA) which recognize the target sequence (here SEQ ID NO: 1) and subsequently trigger their degradation by a poorly understood pathway. In general siRNA duplexes are shorter than 30 nucleotides, because longer stretches of dsRNA activate the PKR pathway in mammalian cells which results in a global a-specific shut-down of protein synthesis. The preparation and gene therapy vectors for the intracellular expression of siRNAs duplexes is disclosed in WO0244321 which is herein incorporated by reference. In another particular embodiment RNA aptamers can be used for the manufacture of a medicament for treatment of pathological angiogenesis. Said RNA aptamers can be generated against prominin-1 (SEQ ID NO: 2). Recently, RNA aptamers have been used as therapeutic reagents for their ability to disrupt protein function. Selection of aptamers in vitro allows rapid isolation of extremely rare RNAs that have high specificity and affinity for specific proteins. Exemplary RNA aptamers are described in U.S. Pat. No. 5,270,163 to Gold et al., Ellington and Szostak, “In vitro Selection of RNA Molecules That Bind Specific Ligands,” Nature 346: 818-822 (1990), and Tuerk and Gold, “Systematic Evolution of Ligands by Exponential Enrichment: RNA Ligands to Bacteriophage T4 DNA Polymerase,” Science 249: 505-510 (1990). Unlike antisense compounds, whose targets are one dimensional lattices, RNA aptamers can bind to the three dimensional surfaces of a protein. Moreover, RNA aptamers can frequently discriminate finely among discrete functional sites of a protein (Gold et al., “Diversity of Oligonucleotide Functions,” Annu. Rev. Biochem. 64: 763-797 (1995)). As research and therapeutic reagents, aptamers not only have the combined advantages of antibodies and small molecular mass drugs, but in vivo production of RNA aptamers also can be controlled genetically Such RNA expressing genes are usually smaller than protein-coding genes and can be inserted into gene therapy vectors.

The term ‘pathological angiogenesis’ refers to the excessive formation and growth of blood vessels during the maintenance and the progression of several disease states. Examples where pathological angiogenesis can occur are blood vessels (atherosclerosis, hemangioma, hemangioendothelioma), bone and joints (rheumatoid arthritis, synovitis, bone and cartilage destruction, osteomyelitis, pannus growth, osteophyte formation, neoplasms and metastasis), skin (warts, pyogenic granulomas, hair growth, Kaposi's sarcoma, scar keloids, allergic oedema, neoplasms), liver, kidney, lung, ear and other epithelia (inflammatory and infectious processes (including hepatitis, glomerulonephritis, pneumonia), asthma, nasal polyps, otitis, transplantation, liver regeneration, neoplasms and metastasis), uterus, ovary and placenta (dysfunctional uterine bleeding (due to intrauterine contraceptive devices), follicular cyst formation, ovarian hyperstimulation syndrome, endometriosis, neoplasms), brain, nerves and eye (retinopathy of prematurity, diabetic retinopathy, choroidal and other intraocular disorders, leukomalacia, neoplasms and metastasis), heart and skeletal muscle due to work overload, adipose tissue (obesity), endocrine organs (thyroiditis, thyroid enlargement, pancreas transplantation), hematopoiesis (AIDS (Kaposi), hematologic malignancies (leukemias, etc.), tumour induced new blood vessels.

The term ‘medicament to treat’ relates to a composition comprising molecules as described above and a pharmaceutically acceptable carrier or excipient (both terms can be used interchangeably) to treat diseases as indicated above. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. The ‘medicament’ may be administered by any suitable method within the knowledge of the skilled man. The preferred route of administration is parenterally. In parental administration, the medicament of this invention will be formulated in a unit dosage injectable form such as a solution, suspension or emulsion, in association with the pharmaceutically acceptable excipients as defined above. However, the dosage and mode of administration will depend on the individual. Generally, the medicament is administered so that the protein, peptide, antibody, small molecule, ribozyme, RNA aptamer, anti-sense nucleic acid or siRNA of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute.

In another embodiment antibodies or functional fragments thereof can be used for the manufacture of a medicament for the treatment of the above mentioned disorders. As a non-limiting example there are the antibodies described in U.S. Pat. No. 5,843,633. In a specific embodiment said antibodies are humanized (Rader et al., 2000, J. Biol. Chem. 275, 13668) and more specifically human antibodies are used to manufacture a medicament to treat pathological angiogenesis. In yet another specific embodiment antibodies derived from camelids are used to manufacture a medicament to treat pathological angiogenesis.

Another aspect of administration for treatment is the use of gene therapy to deliver the above mentioned anti-sense gene or functional parts of the prominin-1 gene or a ribozyme directed against the prominin-1 mRNA or a functional part thereof or RNA aptamers or siRNAs. Gene therapy means the treatment by the delivery of therapeutic nucleic acids to patient's cells. This is extensively reviewed in Lever and Goodfellow 1995; Br. Med Bull., 51, 1-242; Culver 1995; Ledley, F. D. 1995. Hum. Gene Ther. 6, 1129. To achieve gene therapy there must be a method of delivering genes to the patient's cells and additional methods to ensure the effective production of any therapeutic genes. There are two general approaches to achieve gene delivery; these are non-viral delivery and virus-mediated gene delivery.

The invention also provides methods for identifying compounds or molecules which bind on prominin-1 and prevent or suppress pathological angiogenesis. With “suppression” it is understood that said suppression can occur for at least 20%, 30%, 30%, 50%, 60%, 70%, 80%, 90% or even 100%.

Thus in another embodiment the invention provides a method to identify molecules that comprise a region that specifically binds to prominin-1 comprising: (1) exposing prominin-1 or nucleic acids encoding prominin-1 to at least one molecule whose ability to suppress or prevent pathological angiogenesis is sought to be determined, (2) determining binding or hybridising of said molecule(s) to prominin-1 or nucleic acids encoding prominin-1, and (3) monitoring said pathological angiogenesis when administering said molecules as a medicament.

The latter method is also referred to as ‘drug screening assay’ or ‘bioassay’ and typically include the step of screening a candidate/test compound or agent for the ability to interact with prominin-1. Candidate compounds or agents, which have this ability, can be used as drugs to combat or prevent pathological conditions of angiogenesis. Candidate/test compounds are described herein before and are for example RNA aptamers, others are small molecules, e.g. small organic molecules, and other drug candidates can be obtained, for example, from combinatorial and natural product libraries as described above. Typically, the assays are cell-free assays which include the steps of combining prominin-1 and a candidate/test compound (molecule), e.g., under conditions which allow for interaction of (e.g. binding of) the candidate/test compound with prominin-1 to form a complex, and detecting the formation of a complex, in which the ability of the candidate compound to interact with prominin-1 is indicated by the presence of the candidate compound in the complex. Formation of complexes between prominin-1 and the candidate compound can be quantitated, for example, using standard immunoassays. The prominin-1 employed in such a test may be free in solution, affixed to a solid support, borne on a cell surface, or located extracellularly or even intracellularly.

To perform the above described drug screening assays, it is feasible to immobilize prominin-1 or its (their) target molecule(s) to facilitate separation of complexes from uncomplexed forms of one or both of the proteins, as well as to accommodate automation of the assay. Interaction (e.g., binding of) of prominin-1 to a target molecule, can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, prominin-1-His tagged can be adsorbed onto Ni-NTA microtitre plates, or prominin-1-ProtA fusions adsorbed to IgG, which are then combined with the cell lysates (e.g., ³⁵S-labeled) and the candidate compound, and the mixture incubated under conditions conducive to complex formation (e.g., at physiological conditions for salt and pH). Following incubation, the plates are washed to remove any unbound label, and the matrix immobilized and radiolabel determined directly, or in the supernatant after the complexes are dissociated. Alternatively, the complexes can be dissociated from the matrix, separated by SDS-PAGE, and the level of prominin-1-binding protein found in the bead fraction quantitated from the gel using standard electrophoretic techniques. Other techniques for immobilizing protein on matrices can also be used in the drug screening assays of the invention. For example, prominin-1 can be immobilized utilizing conjugation of biotin and streptavidin. Biotinylated prominin-1 can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Another technique for drug screening which provides for high throughput screening of compounds having suitable binding affinity to prominin-1 is described in detail in “Determination of Amino Acid Sequence Antigenicity” by Geysen H N, WO 84/03564, published on 13 Sep. 1984. In summary, large numbers of different small peptide test compounds are synthesized on a solid substrate, such as plastic pins or some other surface. The protein test compounds are reacted with fragments of prominin-1 and washed. Bound prominin-1 is then detected by methods well known in the art. Purified prominin-1 can also be coated directly onto plates for use in the aforementioned drug screening techniques. Alternatively, non-neutralizing antibodies can be used to capture the peptide and immobilize it on a solid support. This invention also contemplates the use of competitive drug screening assays in which neutralizing antibodies capable of binding prominin-1 specifically compete with a test compound for binding prominin-1. In this manner, the antibodies can be used to detect the presence of any protein, which shares one or more antigenic determinants with prominin-1.

### EXAMPLES

1. Generation of a prominin-1 Knock-Out Mice

To study the in vivo role of PROM-1, PROM-1 (prominin-1) deficient mice were generated. Targeted inactivation of the PROM-1 gene was achieved by deletion of exon 2 (containing the start codon). Briefly, a genomic BAC (bacterial artificial chromosome) containing the murine PROM-1 ¹ was obtained from Research Genetics Inc (Huntsville, Ala.) after screening by PCR and hybridization. Mapping of the murine PROM-1 homologue gene revealed that the first exon, which is 79 bp long, is separated from the second exon by an approximately 8 kb intron. It is the second exon (376 bp long) that contains the startcodon ATG. A BamHI fragment of 11.5 kb containing exon 2 was subcloned into the pUC18 plasmid. A targeting vector for inactivation of the PROM-1 gene, pPNT.PROM-1^{null}, was constructed consisting of, from 5′ to 3′: 1.2 kb of 5′ homology comprising the end of intron 1; a loxP-flanked neomycin gene; 5.5 kb from intron 2 as 3′-homology; and a thymidine kinase selection cassette outside of the regions of homology for counterselection against random integration events. The integrity of the construct was verified by restriction digestion and sequencing. The linearized targeting vector pPNTPROM-1^{null} was electroporated in R1 ES cells and targeted clones were identifed by appropriate Southern blot analysis and used for morula aggregation to generate PROM-1 deficient chimeric and germline mice. PROM-1 deficient mice were born at the expected Mendelian frequency (˜25% of 450 offspring from PROM-1 heterozygous breeding pairs). They appeared healthy and were fertile, irrespective of their genetic background (backgrounds tested: 50% Swiss/50% 129, 100% 129, 50% C57BI6/50% 129). We anticipated that PROM-1 might play a crucial role in hematopoiesis implying that the PROM-1^{-/-} embryo would die in utero either after the appearance of the primitive hematopoiesis (7.5 days post coitum, site: yolk sac) or at the emergence of the definitive hematopoiesis (12.5 days post coitum, site: fetal liver). Surprisingly, however, embryonic development in PROM-1^{-/-} mice was normal. PROM-1^{-/-} embryos were not rescued by maternal PROM-1, as PROM-1^{-/-} embryos, sired by PROM-1^{+/-} as well as by PROM-1^{-/-} breeding pairs, developed normally. Also postnatal physiological vascular development seemed normal since no vascular defects could be observed in the heart (capillary density is 5810±154 in PROM-1^{+/+} pups versus 5394±179 in PROM-1^{-/-} pups, n=3; p=NS), kidneys, lungs and skeletal muscle during postnatal growth in PROM-1^{-/-} mice.

2. Impaired Pathological Angiogenesis and/or Vasculogenesis in prominin-1 Knock-Out Mice

In order to study the role of PROM-1 in pathological conditions of angiogenesis PROM-1^{-/-} mice and their wild-type littermates are subjected to various murine models of pathological blood vessel formation.

2.1 Ischemic Retinopathy

PROM-1^{-/-} mice and their wild-type littermates were subjected to a mouse model of ischemic retinopathy. In this hyperoxia-induced retinopathy model, neonatal mice (with an immature retinal vasculature) are exposed to hyperoxia, resulting in obliteration of the developing blood vessels supplying oxygen to the retina. When the mice are then returned to normoxia, the retina, distal to the occluded vessels, becomes ischemic, inducing VEGF production and ultimately resulting in reproducible and quantifiable proliferative retinal neovascularization (33, 34). This model, which mimicks to a certain extent the vascular response during retinopathy of prematurity or diabetic retinopathy, may be useful to test the efficacy of (anti)-angiogenic molecules (Pierce EA et al (1995) Proc. Natl. Acad. Sci. 92 (3) 905-9). Mouse pups of 7 days (P7) together with their mothers, are subjected to hyperoxia (75% oxygen) in specially designed oxygen chambers for 5 days, without opening the cages. On P12, the animals are returned to room air until P17, when the retinas are assessed for maximal neovascular response. Anaesthetized mice are perfused through the left ventricle with 1 ml of phosphate buffered saline containing 50 mg of 2×10⁶ molecular weight fluorescein-dextran. The eyes are removed and fixed in 4% paraformaldehyde for 3 (right eye) or 24 (left eye) hrs. Of the right eyes, lenses are removed and peripheral retinas cut to allow flat mounting with glycerol-gelatin. The flat mounted retinas are analyzed by fluorescence microscopy. The left eyes are embedded in paraffin and serial 6 μm sections are cut sagittally throughout the cornea, parallel to the optic nerve, and stained with hematoxylin-eosin. The proliferative neovascular response is quantified by counting the number of new vessels (=tufts) and the number of endothelial cells extending from the internal limiting membrane of the retina into the vitreum on the stained sagittal cross-sections. The angiographic technique using fluorescein-dextran perfusion is used in conjunction with this counting method for rapid screening of retinas or as an alternative grading system for quantitative evaluation. Loss of prominin-1 significantly protected mice against intra-vitreous neovascularization, as evaluated by counting the number of neovascular tufts and endothelial cells (EC) in the vitrous cavity (n=15; p<0.001)

| | N° of tufts in | N° of EC in |
|---|---|---|
| | vitreous cavity | vitreous cavity |
| | (per 10 retinal sections) | (per 10 retinal sections) |
| PROM-1^{-/- +/+} pups | 157.1 ± 13.6 | 286.0 ± 45.1 |
| (n = 15) | | |
| PROM-1^{-/-} pups | 72.5 ± 14.6 | 106.2 ± 22.6 |
| (n = 15) | | |

2.2 Corneal Micropocket Assay

Hydron pellets containing an angiogenic substance (like bFGF or VEGF) are implanted into the corneal stroma adjacent to the temporal limbus. This induces neovascularization of the avascular corneal stroma from day 3 to day 8 after implantation, without substantial corneal edema or inflammation. Like the retinal hypoxia model, it gives a predictable, persistent and aggressive neovascular response, which is dependent on direct stimulation of blood vessels rather than on indirect stimulation by the induction of inflammation ³⁵. The mouse corneal micropocket assay was performed as previously described ³⁶. Hydron-coated sucralfate pellets containing 300 ng of VEGF₁₆₅ were positioned 1 mm from the corneal limbus. Mice deficient for PROM-1 showed a reduced angiogenic response. The length of the newly formed vessels (0.93±0.12 mm in PROM-1^{+/+} mice versus 0.70±0.03 mm in PROM-1^{-/-} mice, n=6; p<0.005) as well as the circumferential neovascularity (6.23±0.55 mm in PROM-1^{+/+} mice versus 3.60±0.32 mm in PROM-1^{-/-} mice, n=6; p<0.005) and the integrated optical density of the vessel area (497±100 in PROM-1^{-/-} mice versus 196±27 in PROM-1^{-/-} mice, n=6; p<0.05) were significantly lower in the PROM-1 mice. Moreover, WT bone marrow transplantation into PROM-1 deficient mice rescued the impaired angiogenic response. The length of the newly formed vessels (0.57±0.03 mm in PROM-1^{+/+} mice versus 0.57±0.03 mm in PROM-1^{-/-} mice, n=6; p<0.005) as well as the circumferential neovascularity (3.52±0.26 mm in PROM-1^{+/+} mice versus 3.29±0.22 mm in PROM-1^{-/-} mice, n=6; p=NS) were identical in both PROM-1 deficient and WT mice after WT bone marrow transplantation.

2.3 Model of Skin Wound Healing

Vascular remodeling was studied in a model of skin wound healing as described before ^{37,38}. For skin wounding, a standardised 15 mm full-thickness skin incision was made on the back of the mice, taking care not to damage the underlying muscle. Wound healing was quantified by daily measuring the width and the length of the wound. New blood vessel formation was analysed on skin sections harvested four days after wounding. Wound healing was significantly impaired in the PROM-1 deficient mice. Both genotypes contained comparable densities of vessels in unwounded skin. However, the number of capillaries infiltrating the wound (185.8±11.1 vessels/mm² in PROM-1^{+/+} mice versus 135.0±12.7 in PROM-1^{-/-} mice, n=5; p<0.05), as well as the number of smooth muscle-coated vessels in the wounded area (58.2±10 vessels/mm² in PROM-1^{+/+} mice versus 28.6±4.022 in PROM-1^{-/-} mice, n=5; p<0.05) were significantly reduced in PROM-1 deficient mice.

2.4 Matrigel Assay

In-growth of capillaries was studied in a matrigel assay performed as described 39. The angiogenic response in the matrigel of PROM-1^{-/-} mice seemed somewhat lower as measured by the hemoglobin content per matrigel implant (137.0±20.4 μg/ml in PROM-1+/+mice versus 112.1±17.6 μg/ml in PROM-1^{-/-} mice; n=15; p=NS). Histological sections of matrigel were then analysed for infiltration of leukocytes and for blood vessel formation after staining for inflammatory cells (CD45) and endothelial cells (CD31), respectively. The number of infiltrating leukocytes did not seem to differ but a reduced blood vessel formation (CD31 positive endothelial cells) was noticed in the matrigel implanted in PROM-1 deficient animals (% of CD31 positive area in matrigel: 0.55±0.08% in PROM-1^{+/+} mice versus 0.26±0.06% in PROM-1^{-/-} mice; n=5; p<0.05).

2.5 Myocardial Infarction Model

Myocardial infarction was performed by ligation of the LAD as described ⁴⁰. After 4 to 7 days, infarcted hearts were used for histological analysis or for immunostaining of thrombomodulin (endothelial cells) or smooth muscle alpha-actin (smooth muscle cells) ³⁸. Morphometric analysis and counting of immunoreactive cells was performed using an image analysis system with KS300 software (Zeiss, Brussels, Belgium). No differences were observed in the number of capillaries at 4 (490.9±65.4 vessels/mm² in PROM-1^{+/+} mice versus 493.3±87.7 vessels/mm² in PROM-1^{-/-} mice; n=3; p=NS) or 7 days (510.6±28.3 vessels/mm² in PROM-1^{+/+} mice versus 507.8±24.6 vessels/mm² in PROM-1^{-/-} mice; n=10; p=NS) after ligation or in the number of SMC covered vessels at 4 (20.3±3.2 vessels/mm² in PROM-1^{+/+} mice versus 23.3±6.73 vessels/mm² in PROM-1^{-/-} mice; n=3; p=NS) or at 7 days (87.6±14.3 vessels/mm² in PROM-1^{+/+} mice versus 76.4±12.2 vessels/mm² in PROM-1^{-/-} mice; n=10; p=NS) in the infarcted area of hearts of PROM-1 deficient mice and wild-type littermates. However, a clear significant difference was observed in the number of infiltrating macrophages at 7 days after ligation (% of Mac3 positive area: 3.75±0.77% in PROM-1^{+/+} mice versus 1.62±0.42% in PROM-1^{-/-} mice; n=10; p<0.05).

2.6 Hind Limb Ischemia Model

Hind limb ischemia is induced as described ⁴¹. Unilateral right or bilateral ligations of the femoral artery and vein (proximal to the popliteal artery) and the cutaneous vessels branching from the caudal femoral artery side branch is be performed and two superficial preexisting collateral arterioles, connecting the femoral and saphenous artery, will be used for analysis. Genetic consequences on post-ischemic revascularization is determined 14 days after ligation, using vascular morphological (histological evaluation of capillary density and SMC-coated vessel density, histological evaluation of myocyte necrosis and regeneration), perfusional (fluorescent microspheres, laser Doppler imaging), and functional (graded treadmill exercise or swim endurance exercise) analyses.

2.7 Tumor Models

The role of PROM-1 is also tested in tumor models. The following mouse models are operational and are used to analyze tumor angiogenesis in vivo: 1) subcutaneous injection of ras-transformed fibroblasts in athymic nude (nu/nu) mice, 2) subcutaneous injection of Lewis lung carcinoma cells in syngenic C57BI6 hosts, and 3) subcutaneous inoculation of rat C6 glioma cells of athymic nude (nu/nu) mice. Five to twenty million of tumor cells are inoculated in the mice and tumor growth is followed up to 30 days. Tumors are measured with calipers and tumor volumes calculated using the formula [π/6×(w1×w2×w2)], where “w1” and “w2” represent the largest and smallest tumor diameter, respectively. Tumor vessel density and size are determined on tissue sections using immunohistochemistry for visualization of endothelial cells (CD-31), in combination with quantitative morphometry of vascular densities and patterning. If necessary, intratumor flow is determined using colored microspheres to quantitate flow across the entire tumor. When WT RAS transformed fibroblasts were injected in PROM-1 deficient and WT nude mice, no difference in tumor weight was seen (tumor weight after 14 days: 0.9±0.1 g in PROM-1+/+hosts versus 1.1±0.3 g in PROM-1^{-/-}hosts; n=7; p<0.05). Blood vessel analysis is being performed. Remarkably, the number of infiltrating leukocytes was significantly reduced in the tumors grown in the PROM-1 deficient mice.

2.8 LPS Induced Venous Thrombosis in Footpad

To study whether PROM-1 is important in inflammatory processes, a chronic inflammation footpad assay was used. Endotoxin (20 μl, E. coli lipopolysaccharide, 5 and 50 μg/ml) was injected into the right footpad of both PROM-1 deficient and WT mice as described (Carmeliet, P. et al (1993) J. Clin. Invest 6: 2756-60). Saline is injected into the left footpad as a control. After 5 days, mice were sacrificed and both right and left footpad were measured with callipers, excised and fixed in 1% paraformaldehyde for 24 hours. Subsequently, footpads were embedded in paraffin and sectioned. Veins are scored on haematoxilin and eosin stained sections for the presence of thrombi. Five days after injecting 50 μg/ml of endotoxin, a decrease in footpad thickness was observed in the PROM-1 deficient compared to their WT controls.

These data clearly indicate a role of AC 133 in pathological vasculogenesis and/or angiogenesis and implicate the use of inhibitors of PROM-1 in therapeutic strategies to inhibit blood vessel formation in various pathological disorders.

### References

1. Miraglia S, Godfrey W, Yin A H, Atkins K, Warnke R, Holden J T, Bray R A, Waller E K, Buck D W. A novel five-transmembrane hematopoietic stem cell antigen: isolation, characterization, and molecular cloning. Blood 1997; 90 (12): 5013-21.
2. Yin A H, Miraglia S, Zanjani E D, Almeida-Porada G, Ogawa M, Leary A G, Olweus J, Kearney J, Buck D W. PROM-1, a novel marker for human hematopoietic stem and progenitor cells. Blood 1997; 90 (12): 5002-12.
3. Gehling U M, Ergun S, Schumacher U, Wagener C, Pantel K, Otte M, Schuch G, Schafhausen P, Mende T, Kilic N and others. In vitro differentiation of endothelial cells from PROM-1-positive progenitor cells. Blood 2000; 95 (10): 3106-12.
4. Boyer M, Townsend L E, Vogel L M, Falk J, Reitz-Vick D, Trevor K T, Villalba M, Bendick P J, Glover J L. Isolation of endothelial cells and their progenitor cells from human peripheral blood. J Vasc Surg 2000; 31 (1 Pt 1): 181-9.
5. Quirici N, Soligo D, Caneva L, Servida F, Bossolasco P, Deliliers G L. Differentiation and expansion of endothelial cells from human bone marrow CD133(+) cells. Br J Haematol 2001; 115 (1): 186-94.
6. Buhring H J, Seiffert M, Bock T A, Scheding S, Thiel A, Scheffold A, Kanz L, Brugger W. Expression of novel surface antigens on early hematopoietic cells. Ann N Y Acad Sci 1999; 872: 25-38; discussion 38-9.
7. Horn P A, Tesch H, Staib P, Kube D, Diehl V, Voliotis D. Expression of PROM-1, a novel hematopoietic precursor antigen, on acute myeloid leukemia cells. Blood 1999; 93 (4): 1435-7.
8. Kratz-Albers K, Zuhlsdorp M, Leo R, Berdel W L, Buchner T, Serve H. Expression of a PROM-1, a novel stem cell marker, on human leukemic blasts lacking CD34-antigen and on a human CD34+ leukemic line:MUTZ-2. Blood 1998; 92 (11): 4485-7.
9. Buhring H J, Seiffert M, Marxer A, Weiss B, Faul C, Kanz L, Brugger W. PROM-1 antigen expression is not restricted to acute myeloid leukemia blasts but is also found on acute lymphoid leukemia blasts and on a subset of CD34+ B-cell precursors. Blood 1999; 94 (2): 832-3.
10. Corbeil D, Roper K, Hannah M J, Hellwig A, Huttner W B. Selective localization of the polytopic membrane protein prominin in microvilli of epithelial cells—a combination of apical sorting and retention in plasma membrane protrusions. J Cell Sci 1999; 112 (Pt 7): 1023-33.
11. Peichev M, Naiyer A J, Pereira D, Zhu Z, Lane W J, Williams M, Oz M C, Hicklin D J, Witte L, Moore M A and others. Expression of VEGFR-2 and PROM-1 by circulating human CD34 (+) cells identifies a population of functional endothelial precursors. Blood 2000; 95 (3): 952-8.
12. Rafii S. Circulating endothelial precursors: mystery, reality, and promise. J Clin Invest 2000; 105 (1): 17-9.
13. Uchida N, Buck D W, He D, Reitsma M J, Masek M, Phan T V, Tsukamoto A S, Gage F H, Weissman I L. Direct isolation of human central nervous system stem cells. Proc Natl Acad Sci USA 2000; 97 (26): 14720-5.
14. Corbeil D, Roper K, Hellwig A, Tavian M, Miraglia S, Watt S M, Simmons P J, Peault B, Buck D W, Huttner W B. The human PROM-1 hematopoietic stem cell antigen is also expressed in epithelial cells and targeted to plasma membrane protrusions. J Biol Chem 2000; 275 (8): 5512-20.
15. Yu Y, Flint A, Dvorin E L, Bischoff J. PROM-1-2, a Novel Isoform of Human PROM-1 Stem Cell Antigen. J Biol Chem 2002; 277 (23): 20711-6.
16. Maw M A, Corbeil D, Koch J, Hellwig A, Wilson-Wheeler J C, Bridges R J, Kumaramanickavel G, John S, Nancarrow D, Roper K and others. A frameshift mutation in prominin (mouse)-like 1 causes human retinal degeneration. Hum Mol Genet 2000; 9 (1): 27-34.
17. Corbeil D, Roper K, Fargeas C A, Joester A, Huttner W B. Prominin: a story of cholesterol, plasma membrane protrusions and human pathology. Traffic 2001; 2 (2): 82-91.
18. Corbeil D, Roper K, Weigmann A, Huttner W B. PROM-1 hematopoietic stem cell antigen: human homologue of mouse kidney prominin or distinct member of a novel protein family? Blood 1998; 91 (7): 2625-6.
19. Miraglia S, Godfrey W, Buck D. A response to PROM-1 hematopoietic stem cell antigen: human homologue of mouse kidney prominin or distinct member of a novel protein family? Blood 1998; 91 (11): 4390-1.
20. Risau W. Mechanisms of angiogenesis. Nature 1997; 386 (6626): 671-4.
21. Asahara T, Murohara T, Sullivan A, Silver M, van der Zee R, Li T, Witzenbichler B, Schatteman G, Isner J M. Isolation of putative progenitor endothelial cells for angiogenesis. Science 1997; 275 (5302): 964-7.
22. Shi Q, Rafii S, Wu M H, Wijelath E S, Yu C, Ishida A, Fujita Y, Kothari S, Mohle R, Sauvage L R and others. Evidence for circulating bone marrow-derived endothelial cells. Blood 1998; 92 (2): 362-7.
23. Takahashi T, Kalka C, Masuda H, Chen D, Silver M, Kearney M, Magner M, Isner J M, Asahara T. Ischemia- and cytokine-induced mobilization of bone marrow-derived endothelial progenitor cells for neovascularization. Nat Med 1999; 5 (4): 434-8.
24. Asahara T, Masuda H, Takahashi T, Kalka C, Pastore C, Silver M, Kearne M, Magner M, Isner J M. Bone marrow origin of endothelial progenitor cells responsible for postnatal vasculogenesis in physiological and pathological neovascularization. Circ Res 1999; 85 (3): 221-8.
25. Asahara T, Takahashi T, Masuda H, Kalka C, Chen D, Iwaguro H, Inai Y, Silver M, Isner J M. VEGF contributes to postnatal neovascularization by mobilizing bone marrow-derived endothelial progenitor cells. Embo J 1999; 18 (14): 3964-72.
26. Bhattacharya V, McSweeney P A, Shi Q, Bruno B, Ishida A, Nash R, Storb R F, Sauvage L R, Hammond W P, Wu M H. Enhanced endothelialization and microvessel formation in polyester grafts seeded with CD34(+) bone marrow cells. Blood 2000; 95 (2): 581-5.
27. Kalka C, Masuda H, Takahashi T, Kalka-Moll W M, Silver M, Kearney M, Li T, Isner J M, Asahara T. Transplantation of ex vivo expanded endothelial progenitor cells for therapeutic neovascularization. Proc Natl Acad Sci USA 2000; 97 (7): 3422-7.
28. Schatteman G C, Hanlon H D, Jiao C, Dodds S G, Christy B A. Blood-derived angioblasts accelerate blood-flow restoration in diabetic mice. J Clin Invest 2000; 106 (4): 571-8.
29. Kawamoto A, Gwon H C, Iwaguro H, Yamaguchi J I, Uchida S, Masuda H, Silver M, Ma H, Kearney M, Isner J M and others. Therapeutic potential of ex vivo expanded endothelial progenitor cells for myocardial ischemia. Circulation 2001; 103(5): 634-7.
30. Kocher A A, Schuster M D, Szabolcs M J, Takuma S, Burkhoff D, Wang J, Homma S, Edwards N M, Itescu S. Neovascularization of ischemic myocardium by human bone-marrow-derived angioblasts prevents cardiomyocyte apoptosis, reduces remodeling and improves cardiac function. Nat Med 2001; 7 (4): 430-6. 31. Vasa M, Fichtlscherer S, Aicher A, Adler K, Urbich C, Martin H, Zeiher A M, Dimmeler S. Number and migratory activity of circulating endothelial progenitor cells inversely correlate with risk factors for coronary artery disease. Circ Res 2001; 89 (1): E1-7.
32. Gill M, Dias S, Hattori K, Rivera M L, Hicklin D, Witte L, Girardi L, Yurt R, Himel H, Rafii S. Vascular trauma induces rapid but transient mobilization of VEGFR2(+)PROM-1 (+) endothelial precursor cells. Circ Res 2001; 88 (2): 167-74.
33. Smith L E, Wesolowski E, McLellan A, Kostyk S K, D'Amato R, Sullivan R, D'Amore P A. Oxygen-induced retinopathy in the mouse. Invest Ophthalmol Vis Sci 1994; 35 (1): 101-11.
34. Wesolowski E, Smith L E. Effect of light on oxygen-induced retinopathy in the mouse. Invest Ophthalmol Vis Sci 1994; 35 (1): 112-9.
35. Kenyon B M, Voest E E, Chen C C, Flynn E, Folkman J, D'Amato R J. A model of angiogenesis in the mouse cornea. Invest Ophthalmol Vis Sci 1996; 37 (8): 1625-32.
36. Asahara T, Chen D, Takahashi T, Fujikawa K, Kearney M, Magner M, Yancopoulos G D, Isner J M. Tie2 receptor ligands, angiopoietin-1 and angiopoietin-2, modulate VEGF—induced postnatal neovascularization. Circ Res 1998; 83 (3): 233-40.
37. Frank S, Hubner G, Breier G, Longaker M T, Greenhalgh D G, Werner S. Regulation of vascular endothelial growth factor expression in cultured keratinocytes. Implications for normal and impaired wound healing. J Biol Chem 1995; 270 (21): 12607-13.
38. Carmeliet P, Moons L, Luttun A, Vincenti V, Compernolle V, De Mol M, Wu Y, Bono F, Devy L, Beck H and others. Synergism between vascular endothelial growth factor and placental growth factor contributes to angiogenesis and plasma extravasation in pathological conditions. Nat Med 2001; 7 (5): 575-83.
39. Passaniti A, Taylor R M, Pili R, Guo Y, Long P V, Haney J A, Pauly R R, Grant D S, Martin G R. A simple, quantitative method for assessing angiogenesis and antiangiogenic agents using reconstituted basement membrane, heparin, and fibroblast growth factor. Lab Invest 1992; 67 (4): 519-28.
40. Heymans S, Luttun A, Nuyens D, Theilmeier G, Creemers E, Moons L, Dyspersin G D, Cleutjens J P, Shipley M, Angellilo A and others. Inhibition of plasminogen activators or matrix metalloproteinases prevents cardiac rupture but impairs therapeutic angiogenesis and causes cardiac failure. Nat Med 1999; 5 (10): 1135-42.
41. Luttun A, Tjwa M, Moons L, Wu Y, Angelillo-Scherrer A, Liao F, Nagy J A, Hooper A, Priller J, De Klerck B and others. Revascularization of ischemic tissues by PIGF treatment, and inhibition of tumor angiogenesis, arthritis and atherosclerosis by anti-Flt1. Nat Med 2002; 8 (8): 831-40.
42. Gale N W, Yancopoulos G D. Growth factors acting via endothelial cell-specific receptor tyrosine kinases: VEGFs, angiopoietins, and ephrins in vascular development. Genes Dev 1999; 13 (9): 1055-66.
43. Carmeliet P. Mechanisms of angiogenesis and arteriogenesis. Nat Med 2000; 6 (4): 389-95.
44. Breiteneder-Geleff S, Soleiman A, Kowalski H, Horvat R, Amann G, Kriehuber E, Diem K, Weninger W, Tschachler E, Alitalo K and others. Angiosarcomas express mixed endothelial phenotypes of blood and lymphatic capillaries: podoplanin as a specific marker for lymphatic endothelium. Am J Pathol 1999; 154 (2): 385-94.
45. Wigle J T, Oliver G. Prox1 function is required for the development of the murine lymphatic system. Cell 1999; 98 (6): 769-78.
46. Jackson D G, Prevo R, Clasper S, Banerji S. LYVE-1, the lymphatic system and tumor lymphangiogenesis. Trends Immunol 2001; 22 (6): 317-21.
47. Veikkola T, Jussila L, Makinen T, Karpanen T, Jeltsch M, Petrova T V, Kubo H, Thurston G, McDonald D M, Achen M G and others. Signalling via vascular endothelial growth factor receptor-3 is sufficient for lymphangiogenesis in transgenic mice. Embo J 2001; 20 (6): 1223-31.
48. Sleeman J P, Krishnan J, Kirkin V, Baumann P. Markers for the lymphatic endothelium: in search of the holy grail? Microsc Res Tech 2001; 55 (2): 61-9.
49. Witte M H, Bernas M J, Martin C P, Witte C L. Lymphangiogenesis and lymphangiodysplasia: from molecular to clinical lymphology. Microsc Res Tech 2001; 55 (2): 122-45.
50. Karpanen T, Egeblad M, Karkkainen M J, Kubo H, Yla-Herttuala S, Jaattela M, Alitalo K. Vascular endothelial growth factor C promotes tumor lymphangiogenesis and intralymphatic tumor growth. Cancer Res 2001; 61 (5): 1786-90.
51. Mandriota S J, Jussila L, Jeltsch M, Compagni A, Baetens D, Prevo R, Banerji S, Huarte J, Montesano R, Jackson D G and others. Vascular endothelial growth factor-C-mediated lymphangiogenesis promotes tumour metastasis. Embo J 2001; 20 (4): 672-82.
52. Skobe M, Hawighorst T, Jackson D G, Prevo R, Janes L, Velasco P, Riccardi L, Alitalo K, Claffey K, Detmar M. Induction of tumor lymphangiogenesis by VEGF-C promotes breast cancer metastasis. Nat Med 2001; 7 (2): 192-8.
53. Stacker S A, Caesar C, Baldwin M E, Thornton G E, Williams R A, Prevo R, Jackson D G, Nishikawa S, Kubo H, Achen M G. VEGF-D promotes the metastatic spread of tumor cells via the lymphatics. Nat Med 2001; 7 (2): 186-91.
54. Alitalo K, Carmeliet P. Molecular mechanisms of lymphangiogenesis in health and disease. Cancer Cell 2002; 1 (3): 219-27.
55. Oliver G, Detmar M. The rediscovery of the lymphatic system: old and new insights into the development and biological function of the lymphatic vasculature. Genes Dev 2002; 16 (7): 773-83.
56. Schneider M, Othman-Hassan K, Christ B, Wilting J. Lymphangioblasts in the avian wing bud. Dev Dyn 1999; 216 (4-5): 311-9.
57. Kubo H, Cao R, Brakenhielm E, Makinen T, Cao Y, Alitalo K. Blockade of vascular endothelial growth factor receptor-3 signaling inhibits fibroblast growth factor-2-induced lymphangiogenesis in mouse cornea. Proc Natl Acad Sci USA 2002; 99 (13): 8868-73.
58. Folkman J. A mechanism for regulation of lymphangiogenesis independent of angiogenesis. Bethesda; 2002.
59. Baldwin M, Catimel B, Nice E C, Roufail S, Hall N E, Stenvers K L, Karkkainen M J, Alitalo K, Stacker S A, M. G. A. The Specificity of Receptor Binding by Vascular Endothelial Growth Factor-D Is Different in Mouse and Man. J. Biol. Chem. 2001; 276 (22): 19166-19171.
60. Boardman K C, Swartz M A. Lymphangiogenesis in a mouse tail model. 2001. p 671-672.
61. Ikomi F, Ohhhashi T. Immunohistochemical expression of VEGF receptors on new vascularization of collecting lymphatics in mice. In: Editore M, editor; 2001; Sidney, Australia. p 387-390.
62. Reynolds B A, Weiss S. Generation of neurons and astrocytes from isolated cells of the adult mammalian central nervous system. Science 1992; 255 (5052): 1707-10.
63. Gage F H. Mammalian neural stem cells. Science 2000; 287 (5457): 1433-8.
64. Yamamoto S, Yamamoto N, Kitamura T, Nakamura K, Nakafuku M. Proliferation of parenchymal neural progenitors in response to injury in the adult rat spinal cord. Exp Neurol 2001; 172 (1): 115-27.
65. Cao Q, Benton R L, Whittemore S R. Stem cell repair of central nervous system injury. J Neurosci Res 2002; 68 (5): 501-10.
66. Arvidsson A, Collin T, Kirik D, Kokaia Z, Lindvall O. Neuronal replacement from endogenous precursors in the adult brain after stroke. Nat Med 2002; 8 (9): 963-70.
67. Kempermann G, Kuhn H G, Gage F H. Genetic influence on neurogenesis in the dentate gyrus of adult mice. Proc Natl Acad Sci USA 1997; 94 (19): 10409-14.
68. Kuhn H G, Palmer T D, Fuchs E. Adult neurogenesis: a compensatory mechanism for neuronal damage. Eur Arch Psychiatry Clin Neurosci 2001; 251 (4): 152-8.
69. Gritti A, Parati E A, Cova L, Frolichsthal P, Galli R, Wanke E, Faravelli L, Morassutti D J, Roisen F, Nickel D D and others. Multipotential stem cells from the adult mouse brain proliferate and self-renew in response to basic fibroblast growth factor. J Neurosci 1996; 16 (3): 1091-100.
70. Takagi Y, Mitsui A, Nishiyama A, Nozaki K, Sono H, Gon Y, Hashimoto N, Yodoi J. Overexpression of thioredoxin in transgenic mice attenuates focal ischemic brain damage. Proc Natl Acad Sci USA 1999; 96 (7): 4131-6.
71. Tamaki S, Eckert K, He D, Sutton R, Doshe M, Jain G, Tushinski R, Reitsma M, Harris B, Tsukamoto A and others. Engraftment of sorted/expanded human central nervous system stem cells from fetal brain. J Neurosci Res 2002; 69 (6): 976-86.
72. Wu S, Suzuki Y, Kitada M, Kataoka K, Kitaura M, Chou H, Nishimura Y, Ide C. New method for transplantation of neurosphere cells into injured spinal cord through cerebrospinal fluid in rat. Neurosci Lett 2002; 318 (2): 81-4.
73. Lang-Lazdunski L, Matsushita K, Hirt L, Waeber C, Vonsattel J P, Moskowitz M A, Dietrich W D. Spinal cord ischemia. Development of a model in the mouse. Stroke 2000; 31 (1): 208-13.
74. Rapalino O, Lazarov-Spiegler O, Agranov E, Velan G J, Yoles E, Fraidakis M, Solomon A, Gepstein R, Katz A, Belkin M and others. Implantation of stimulated homologous macrophages results in partial recovery of paraplegic rats. Nat Med 1998; 4 (7): 814-21.

1. A method of using a molecule that inhibits the expression and/or activity of prominin-1 for treatment of pathological angiogenesis.

2. A method of using a molecule according to claim 1 wherein said molecule is selected from the group consisting of an antibody or any fragment thereof, a small molecule, an RNA aptamer, a peptide, a ribozyme, anti-sense nucleic acids and siRNA.

3. A method to identify molecules that comprise a region that specifically binds to prominin-1 comprising:
exposing prominin-1 or nucleic acids encoding prominin-1 to at least one molecule whose ability to suppress or prevent pathological angiogenesis is sought to be determined.
determining binding or hybridizing of said molecule(s) to prominin-1 or nucleic acids encoding prominin-1, and
monitoring said pathological angiogenesis when administering said molecules as a medicament.


SEQUENCE LISTING (2 sequences)

SEQ ID NO: 1 - DNA, 3794 bp, Homo sapiens
  CDS (38)..(2635)
ccaagttctacctcatgtttggaggatcttgctagctatggccctcgtactcggctccctgttgctgctggggctgtgcgggaactccttttcaggagggcagccttcatccacagatgctcctaaggcttggaattatgaattgcctgcaacaaattatgagacccaagactcccataaagctggacccattggcattctctttgaactagtgcatatctttctctatgtggtacagccgcgtgatttcccagaagatactttgagaaaattcttacagaaggcatatgaatccaaaattgattatgacaagccagaaactgtaatcttaggtctaaagattgtctactatgaagcagggattattctatgctgtgtcctggggctgctgtttattattctgatgcctctggtggggtatttcttttgtatgtgtcgttgctgtaacaaatgtggtggagaaatgcaccagcgacagaaggaaaatgggcccttcctgaggaaatgctttgcaatctccctgttggtgatttgtataataataagcattggcatcttctatggttttgtggcaaatcaccaggtaagaacccggatcaaaaggagtcggaaactggcagatagcaatttcaaggacttgcgaactctcttgaatgaaactccagagcaaatcaaatatatattggcccagtacaacactaccaaggacaaggcgttcacagatctgaacagtatcaattcagtgctaggaggcggaattcttgaccgactgagacccaacatcatccctgttcttgatgagattaagtccatggcaacagcgatcaaggagaccaaagaggcgttggagaacatgaacagcaccttgaagagcttgcaccaacaaagtacacagcttagcagcagtctgaccagcgtgaaaactagcctgcggtcatctctcaatgaccctctgtgcttggtgcatccatcaagtgaaacctgcaacagcatcagattgtctctaagccagctgaatagcaaccctgaactgaggcagcttccacccgtggatgcagaacttgacaacgttaataacgttcttaggacagatttggatggcctggtccaacagggctatcaatcccttaatgatatacctgacagagtacaacgccaaaccacgactgtcgtagcaggtatcaaaagggtcttgaattccattggttcagatatcgacaatgtaactcagcgtcttcctattcaggatatactctcagcattctctgtttatgttaataacactgaaagttacatccacagaaatttacctacattggaagagtatgattcatactggtggctgggtggcctggtcatctgctctctgctgaccctcatcgtgattttttactacctgggcttactgtgtggcgtgtgcggctatgacaggcatgccaccccgaccacccgaggctgtgtctccaacaccggaggcgtcttcctcatggttggagttggattaagtttcctcttttgctggatattgatgatcattgtggttcttacctttgtctttggtgcaaatgtggaaaaactgatctgtgaaccttacacgagcaaggaattattccgggttttggatacaccctacttactaaatgaagactgggaatactatctctctgggaagctatttaataaatcaaaaatgaagctcacttttgaacaagtttacagtgactgcaaaaaaaatagaggcacttacggcactcttcacctgcagaacagcttcaatatcagtgaacatctcaacattaatgagcatactggaagcataagcagtgaattggaaagtctgaaggtaaatcttaatatctttctgttgggtgcagcaggaagaaaaaaccttcaggattttgctgcttgtggaatagacagaatgaattatgacagctacttggctcagactggtaaatcccccgcaggagtgaatcttttatcatttgcatatgatctagaagcaaaagcaaacagtttgcccccaggaaatttgaggaactccctgaaaagagatgcacaaactattaaaacaattcaccagcaacgagtccttcctatagaacaatcactgagcactctataccaaagcgtcaagatacttcaacgcacagggaatggattgttggagagagtaactaggattctagcttctctggattttgctcagaacttcatcacaaacaatacttcctctgttattattgaggaaactaagaagtatgggagaacaataataggatattttgaacattatctgcagtggatcgagttctctatcagtgagaaagtggcatcgtgcaaacctgtggccaccgctctagatactgctgttgatgtctttctgtgtagctacattatcgaccccttgaatttgttttggtttggcataggaaaagctactgtatttttacttccggctctaatttttgcggtaaaactggctaagtactatcgtcgaatggattcggaggacgtgtacgatgatgttgaaactatacccatgaaaaatatggaaaatggtaataatggttatcataaagatcatgtatatggtattcacaatcctgttatgacaagcccatcacaacattgatagctgatgttgaaactgcttgagcatcaggatactcaaagtggaaaggatcacagatttttggtagtttctgggtctacaaggactttccaaatccaggagcaacgccagtggcaacgtagtgactcaggcgggcaccaaggcaacggcaccattggtctctgggtagtgctttaagaatgaacacaatcacgttatagtccatggtccatcactattcaaggatgactccctcccttcctgtctatttttgttttttacttttttacactgagtttctatttagacactacaacatatggggtgtttgttcccattggatgcatttctatcaaaactctatcaaatgtgatggctagattctaacatattgccatgtgtggagtgtgctgaacacacaccagtttacaggaaagatgcattttgtgtacagtaaacggtgtatataccttttgttaccacagagttttttaaacaaatgagtattataggactttcttctaaatgagctaaataagtcaccattgacttcttggtgctgttgaaaataatccattttcactaaaagtgtgtgaaacctacagcatattcttcacgcagagattttcatctattatactttatcaaagattggccatgttccacttggaaatggcatgcaaaagccatcatagagaaacctgcgtaactccatctgacaaattcaaaagagagagagagatcttgagagagaaatgctgttcgttcaaaagtggagttgttttaacagatgccaattacggtgtacagtttaacagagttttctgttgcattaggataaacattaattggagtgcagctaacatgagtatcatcagactagtatcaagtgttctaaaatgaaatatgagaagatcctgtcacaattcttagatctggtgtccagcatggatgaaacctttgagtttggtccctaaatttgcatgaaagcacaaggtaaatattcatttgcttcaggagtttcatgttggatctgtcattatcaaaagtgatcagcaatgaagaactggtcggacaaaatttaacgttgatgtaatggaattccagatgtaggcattccccccaggtcttttcatgtgcagattgcagttctgattcatttgaataaaaaggaacttgg

SEQ ID NO: 2 - protein, 865 aa, Homo sapiens
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH